(19) United States
(12) Patent Application Publication
(10) Pub. No.: US 2008/0175834 A1
(43) Pub. Date: Jul. 24, 2008

(54) Carboxypertidase U (Cpu) Mutants

(75) Inventors: Philippe Cronet, Molndal (SE); Wolfgang Knecht, Molndal (SE); Cecilia Ann-Christin Malmborg Hager, Lund (SE); Mats Andersson, Lund (SE); Christina Furebring, Lund (SE)

Correspondence Address: ASTRAZENECA R&D BOSTON, 35 GATEHOUSE DRIVE, WALTHAM, MA, 02451-1215, US

(73) Assignee: AstraZenca AB, Sodertalje (SE)

(21) Appl. No.: 10/579,117

(22) PCT Filed: Nov. 10, 2004
(86) PCT No.: PCT/GB2004/004731

§ 371 Date: Nov. 2, 2007

(30) Foreign Application Priority Data
Nov. 15, 2003 (GB) 0326676.4

(51) Int. Cl. A61K 38/48; C12N 9/48; C12N 15/11; C12N 15/00; A61P 43/00; C12N 5/06; C12P 21/04

(52) U.S. Cl. 424/94.63; 435/212; 536/23.2; 435/320.1; 435/69.1; 530/387.9; 435/188

(57) ABSTRACT

This invention relates to mutant forms of carboxypeptidase U with increased thermal stability relative to wild-type. In addition to the individual thermal stabilizing mutations identified (at positions 166, 204, 219, 230, 251, 315), the inventors have identified a region (S327-H357) that is crucial to the stability of CPU. The invention relates to nucleic acid encoding such mutant forms and the polypeptides encoded thereby. The invention also relates to methods and materials for making CPU mutants with increased thermal stability relative to wild-type and their use, for example to produce crystals of CPU or proCPU for 3-dimensional structure determination, or in therapy.

This invention relates to mutant forms of carboxypeptidase U with increased thermal stability relative to wild-type. In addition to individual thermal stabilizing mutations identified herein, the inventors have identified a region (S327-H357) that is crucial to the stability of CPU. The invention relates to nucleic acid encoding such mutant forms and the polypeptides encoded thereby. The invention also relates to methods and materials for making CPU mutants with increased thermal stability relative to wild-type and their use, for example to produce crystals of CPU or proCPU for 3-dimensional structure determination, or in therapy.

Carboxypeptidase U (CPU, EC 3.4.17.20) is a Zn metallopeptidase that circulates in plasma in a zymogen form, proCPU. CPU has also been named active thrombin-activatable fibrinolysis inhibitor (TAFIa), plasma carboxypeptidase B or carboxypeptidase R. The term CPU is used herein. ProCPU is converted to CPU during coagulation or fibrinolysis by the action of thrombin, the thrombin-thrombomodulin complex or plasmin. CPU is a very unstable enzyme (indeed, the U in CPU stands for unstable). CPU cleaves basic amino acids at the carboxy-terminal of fibrin fragments. The loss of carboxy-terminal lysines and thereby of lysine binding sites for plasminogen and t-PA then serves to downregulate fibrinolysis

The deduced amino acid sequence of the protein reveals a primary translation product very similar to tissue-type carboxypeptidases A and B. Eaton et al (J Biol Chem. 266(32):21833-8,1991) cloned the cDNA for human proCPU. The predicted 423 amino acid protein, consists of a 22-amino acid signal peptide, a 92-amino acid activation peptide, and a 309-amino acid catalytic domain. Tsai and Drayna (Genomics. 14:549-550,1992) demonstrated that the gene is located on human chromosome 13. The gene was regionalized by Vanhoof et al. (Genomics 38:454-455, 1996) using fluorescence in situ hybridisation to 13q14.11. The four common natural allelic forms found in the human population are (in preproCPU numbering) T169/T341; T169/I347; A169/T347 and A169/I347 (Schneider et al. J. Biol. Chem. 277(2):1021-1030, 2002). In this publication it is shown that the two I347 containing variants are 2-fold more stable than the two other variants.

For the purpose of this application the A169/T347 variant is taken as wild-type. The sequence of this common polymorphism is shown in SEQ ID NO: 1 and 2.

A possible role for CPU is in the inhibition of the activation of plasminogen to produce plasmin, an enzyme which catalyzes the degradation of fibrin. A balance between the activities of the coagulation and fibrinolysis cascades is essential to protect the organism from excessive blood loss upon injury and to maintain blood fluidity within the vascular system. Imbalances are characterized by either bleeding or thrombotic tendencies, the latter of which are manifested as heart attacks and strokes. Inhibition of CPU to accelerate fibrinolysis could be a treatment for thromboembolic disorders.

Native proCPU has been purified from human plasma and recombinant proCPU has been produced in stably transduced mammalian cell lines or using the baculovirus vector expression system in insect cells (Schneider et al., J. Biol. Chem. 277(2):1021-1030, 2002; Strömqvist et al., Thrombosis and Haemostasis 85:12-17, 2001; Zhao et al., Thrombosis and Haemostasis. 80(6):949-55, 1998, Strömqvist et al., Clinica Chimica Acta. 347:49-59, 2004).

Although crystal structures for other carboxypeptidases, e.g., carboxypeptidase B (CPB) have been solved. The crystal structure of CPU has not yet been deduced. The difficulties of crystallizing CPU are believed to be due to the relative instability of the enzyme, in combination with a relatively low solubility (<0.3 mg/mL).

Because of its prominent bridging function between coagulation and fibrinolysis, the development of CPU inhibitors as pro-fibrinolytic agents is an attractive concept (Zirlik, Thromb Haemost. 91(3):420-2, 2004; Lazoura et al., Chem Biol. 9(10):1129-39, 2002). But the structural characterization of CPU, and use of this knowledge, for drug design has been severely hampered by its instability. Only a 3-dimensional model of human proCPU based on the structure of human pancreas procarboxypeptidase B has been published recently by Barbosa Pereira et al., (J Mol Biol. 321(3):537-47, 2002).

The present invention is not concerned with natural allelic forms of preproCPU, proCPU or CPU, but to engineered mutant forms that have enhanced thermal stability (in vitro half-life) relative to the natural allelic forms.

WO 02/099098 (American Diagnostica) teaches a method to prepare stable TAFIa (CPU) by activating it in an essentially calcium free environment with a protease that cleaves TAFI to TAFIa and keeping it in an essentially calcium free environment.

There is a need in the art for a more thermostable form of CPU that is more amenable to crystallization.

### BRIEF DESCRIPTION OF THE INVENTION

The present invention provides mutant forms of carboxypeptidase U (CPU) that are more thermostable than any of the four naturally occurring wild-type allelic forms of CPU. The mutations are substitutions of one or more critical amino acids. Preferred substitutions are outlined in Table 1.

TABLE 1
preferred substitutions

| Amino | |
|---|---|
| acid | Substitution |
| K | By one neutral (uncharged) polar residue such as serine, threonine, tyrosine, |
| | asparagine, glutamine, or cysteine; or by a positively charged residue such as |
| | arginine or histidine |
| I | By one neutral (uncharged) polar residue such as serine, threonine, tyrosine, |
| | asparagine, glutamine, or cysteine |
| V | By one non-polar or hydrophobic residue such as alanine, leucine, isoleucine, |
| | proline methionine, phenylalanine or tryptophan |
| Y | By one neutral (uncharged) polar residue such as serine, threonine, |
| | asparagine, glutamine, or cysteine |
| H | By one non-polar or hydrophobic residue such as alanine, leucine, isoleucine, |
| | valine, proline methionine, phenylalanine or tryptophan; or by one neutral |
| | (uncharged) polar residue such as serine, threonine, tyrosine, asparagine, |
| | glutamine, or cysteine; or by a positively charged residue such as lysine or |
| | arginine. |
| S | By one neutral (uncharged) polar residue such as threonine, tyrosine, |
| | asparagine, glutamine, or cysteine |
| N | By one neutral (uncharged) polar residue such as serine, threonine, tyrosine, |
| | glutamine, or cysteine |
| R | By a positively charged residue such as lysine or histidine |

Mutants possessing two or more selected substitutions are considerably more thermostable. The invention also provides nucleic acid molecules that encode such mutant CPU polypeptides, vectors housing such nucleic acids, host cell comprising such nucleic acids, methods for making the mutant CPU polypeptides of the invention, their use in the manufacture of pharmaceutical compositions and their use in therapy, and the use of the polypeptides to make crystal structures of CPU and CPU containing complexes.

### BRIEF DESCRIPTION OF THE FIGURES

FIG. 1.—shows the alignment of human (SEQ ID NO:2), mouse (SEQ ID NO: 12) and rat (SEQ ID NO: 13) preproCPU. The position of mutated amino acids found in human preproCPU and the corresponding amino acids in mouse and rat preproCPU are shaded.

FIG. 2—shows the thermostability of CPU and the CPU mutant HQ determined either using a HPLC (see example 6) or the Hippuricase assay (see example 2). The enzymatic activity at t=0 was arbitrarily set to 100% for each determination. Closed circles: CPU (Hippuricase assay); open circles: HQ (Hippuricase assay); closed triangles: HQ (HPLC assay).

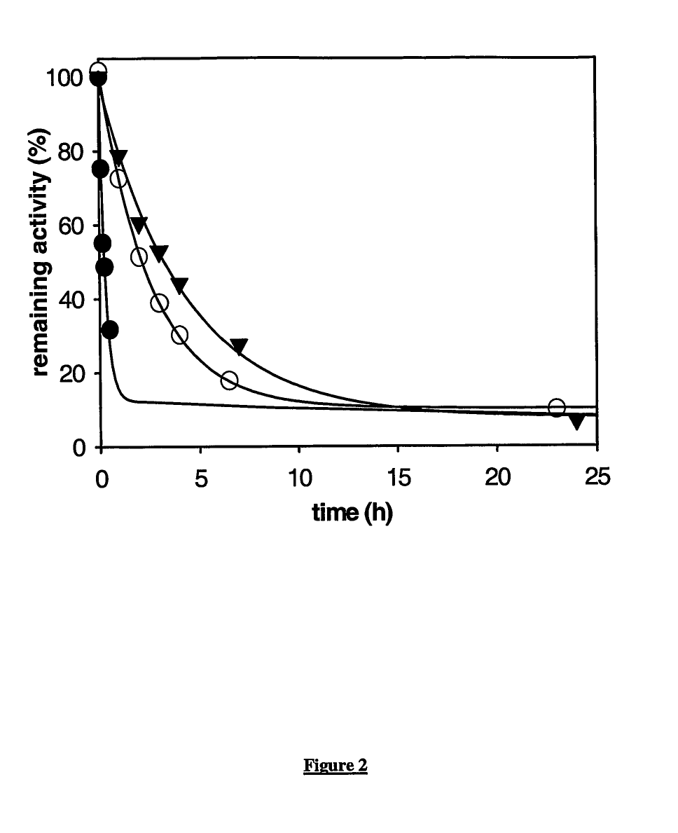

FIG. 3—shows mutants discovered (T½ is 4 fold or more than WT in at least one of the two determinations)

### DETAILED DESCRIPTION OF THE INVENTION

The CPU protein exhibits remarkable evolutionary sequence conservation. Indeed human, rat and mouse preproCPU are either 423 or 422 amino acids in length and possess at least 80% sequence identity.

Reference herein to the mutation position within a CPU polynucleotide in general relate to the position in human preproCPU (SEQ ID NO 1) unless stated otherwise or apparent from the context.

All positions herein of mutations in the human CPU polynucleotide relate to the position in SEQ ID NO 1 unless stated otherwise or apparent from the context.

Reference herein to the mutation position within a CPU polypeptide per se relate to the position in human preproCPU (SEQ ID NO 2) unless stated otherwise or apparent from the context.

Unless otherwise indicated, reference herein to CPU mutants includes CPU mutants with the pro- or prepro-portion, upstream of the mature CPU polypeptide, still attached.

All positions herein of mutation in the human CPU polypeptide relate to the position in SEQ ID NO 2 unless stated otherwise or apparent from the context.

All positions herein of mutation in the mouse CPU polypeptide relate to the position in SEQ ID NO 12 (corresponds to database entries MER06276, GB:AF186188) unless stated otherwise or apparent from the context.

All positions herein of mutation in the rat CPU polypeptide relate to the position in SEQ ID NO 13 (corresponds to database entries MER15161, GB:AB042598) unless stated otherwise or apparent from the context.

Substitution mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The inventors have determined that one or more amino acid substitution mutations at the following positions (relative to wild-type preproCPU depicted in SEQ ID NO: 2): 166, 204, 219, 230, 251, 315, 327, 346, 348, 349, 350, 352, 355 and 357, have greater thermal stability than wild-type protein. In particular, these results have identified a hot-spot region (amino acids 327 to 357, inclusive). This region harbors more than 50% of the stabilising substitution positions within a stretch of less than 10% of SEQ ID No: 2.

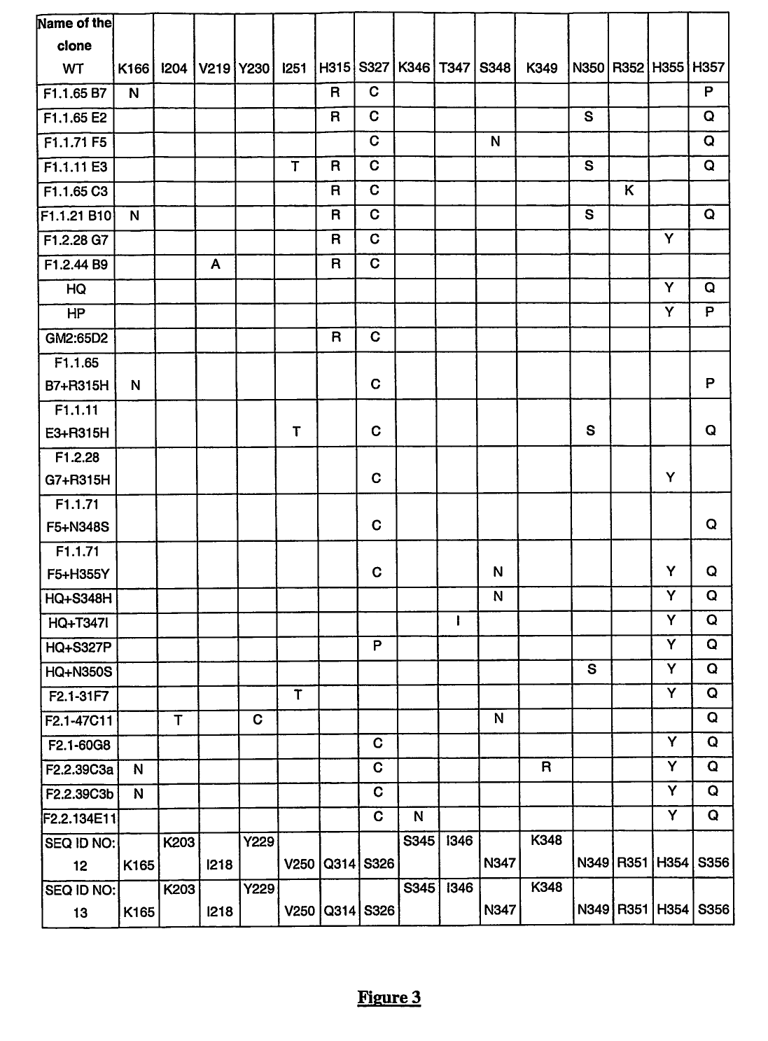

Although the invention is illustrated using human CPU (SEQ ID NO:2), the mutant CPU may be from any mammalian source. FIG. 3 identifies the location of the corresponding substitution position in both mouse (relative to SEQ ID NO:12) and rat (relative to SEQ ID NO:13).

The mutant/variant CPU proteins of the invention have increased stability relative to the wild-type CPU proteins. As noted above, the natural I347 containing variants are approximately 2-fold more stable that the T347 containing variants. As the A169/T347 variant is selected as representing the comparator wild-type protein, by increased stability, as used herein, we refer to an increased in vitro half-life, exhibiting, in increasing order of preference, at least 4-fold, 5-, 10-, 15-, 20-, 25-fold, or more, greater stability that that of the A169/T347 allelic form of native CPU. To measure the half-life, CPU is incubated at 37° C. and at certain time points samples are taken and the remaining enzymatic activity is measured (by either or both HPLC (see example 6) or the Hippuricase assay (see example 2)). Half-life is the time after which 50% of the initial activity is lost. The wild-type CPUs have half-lives of about 7.8-17.8 min (depending on the polymorphism, see Table 2), whereas some of the mutants of the present invention have half-lives in excess of one hour. Those mutants with ½ lives of greater than one hour at 37° C. are of particular use in the various applications disclosed herein.

Amino acids 166 to 357 are located in CPU (i.e. not in the prepro region). By computer modeling of CPU against CPB by the method according to Pereira et al. (J. Mol. Biol. 321: 537-547, 2002), the inventors predict that all the identified mutant sites, except for V219, are located on the surface of the protein. Thus the ‘hotspot’ region (R327-H357) harboring most of the stabilizing mutation sites is believed to be on the protein surface.

In particular embodiments, the mutant forms of CPU with one or more of the following specific substitutions have been made and shown to possess enhanced thermal stability: K166N, I204T, V219A, Y230C, I251T, H315R, S327C, K346N, S348N, K349R, N350S, R352K, H355Y, H357P and H357Q.

The inventors have found that mutant CPU proteins that comprise just one amino acid substitution at an identified location possess enhanced thermal stability. However those with 2 or more, such as 2, 3, 4, 5 or more of the designated substitutions have greater thermal stability than singly substituted mutant forms. Accordingly, in separate embodiments the mutant CPU forms posses one, two, three, four, five, six, seven, eight or more amino acid substitutions relative to the human CPU depicted as SEQ ID NO: 2.

Of the mutants generated, those that include substitution at one or more of the following three sites 327, 355 and 357, were found to be particularly stable.

According to one aspect of the invention there is provided a carboxypeptidase U (CPU) mutant polypeptide having greater thermal stability than the wild-type polypeptide, which mutant possesses an amino acid substitution located at an amino acid residue position relative to SEQ ID NO: 2, selected from the group consisting of: 166, 204, 219, 230, 251, 315 and from within 327 to 357. The term “from within” used in this context, includes positions 327 and 357, and refers to an amino acid substitution on any amino acid from residue 327 to 357, inclusive.

Stipulating the location of the substitution position relative to human CPU allows identification of the corresponding position in CPU from other species, including rat and mouse.

Preferred substitutions are those listed in Table 1. These may include synonymous amino acids within a group, which have sufficiently similar physicochemical properties that substitution between members of the group will preserve the biological function of the molecule.

Although single substitution mutants have enhanced thermal stability, the inventors have found that at least two mutations are required to generate forms with at least 4-fold enhanced thermal stability.

According to a further aspect of the invention there is provided a carboxypeptidase U (CPU) mutant polypeptide having greater thermal stability than the wild-type polypeptide, which mutant possesses at least two amino acid substitutions, at least one of which is located at an amino acid residue position relative to SEQ D NO: 2, selected from the group consisting of: 166, 204, 219, 230, 251, 315 and from within 327 to 357. In a preferred embodiment the second, and optionally additional, substitution is also located at one of the identified positions. In alternative embodiments, the mutant has relative to wild-type CPU, 2, 3, 4, 5, 6, 7, 8, 9, 10, or more substitutions.

According to a further aspect of the invention there is provided a CPU mutant polypeptide with at least one of the amino acid substitution within the amino acid region 327 and 357 inclusive, according to the position in SEQ ID NO: 2.

According to a further aspect of the invention there is provided a CPU mutant polypeptide with an amino acid substitution at one or other of positions S327, H355 or H357, relative to SEQ ID NO: 2, optionally, in combination with at least one other amino acid substitution. In a particular embodiment, this second or further substitution can include another of the three stipulated sites.

According to a further aspect of the invention there is provided a CPU mutant polypeptide with at least one of the following amino acid substitutions: S327C, H355Y or H357Q, relative to SEQ ID NO: 2, optionally, in combination with at least one other substitution mutation. In a particular embodiment, this second or further substitution can include another of the three stipulated substitutions.

In particular embodiments, the amino acid substitution(s) is/are one or more of: K166N, I204T, V219A, Y230C, I251T, H315R, S327C, K346N, S348N, K349R, N350S, R352K, H355Y, H357P or H357Q.

Particular CPU mutants are those with multiple substitutions, in particular mutants that comprise the following combination of substitutions: S327C and H355Y; S327C and H357Q; or H355Y and H357Q.

According to a further aspect of the invention there is provided an isolated polypeptide comprising the amino acid sequence depicted in any of SEQ ID Nos: 17, 18 or 19.

SEQ ID NO: 17 depicts the amino acid sequence of the HQ mutant. SEQ ID NO: 18 depicts the amino acid sequence of the F1.1.71 F5+H355Y mutant. SEQ ID NO: 19 depicts the amino acid sequence of the F2.1-60G8 mutant.

Further aspects of the invention include nucleic acid molecules that encode a mutant CPU of the present invention, vectors, in particular plasmid vectors, which contain such nucleic acids, and host cells comprising nucleic acids that encode the mutant CPUs of the invention.

According to another aspect of the present invention there is provided an isolated nucleic acid molecule comprising a nucleotide sequence that encodes an CPU variant with enhanced thermal stability than the wild-type protein, which variant differs from the wild-type protein in possessing one or more amino acid substitutions located at positions: 166, 204, 219, 230, 251, 315 and from within 327 to 357, relative to the position in SEQ ID NO: 2.

According to a further aspect of the present invention there is provided an isolated nucleic acid molecule comprising a nucleotide sequence that encodes an CPU variant with enhanced thermal stability than the wild-type protein, which variant differs from the wild-type protein in possessing at least two amino acid substitutions, at least one of which is selected from an amino acid located on the surface of the protein selected from positions: 166, 204, 230, 251, 315 and from within 327 to 357, relative to the position in human CPU (SEQ ID NO: 2). Particular substitutions from within the 327-357 region are at positions: 327, 346, 348, 349, 350, 352, 355 and 357.

As used herein, the term “isolated” or “purified” refers to molecules, either nucleic acid or amino acid sequences, that are removed from their natural environment and purified or separated from at least one other component with which they are naturally associated. Also encompassed by this term are molecules that are artificially synthesized and purified away from their synthesis materials. Thus, a polynucleotide is said to be isolated when it is substantially separated from other contaminant polynucleotides or nucleotides.

The introduction of a mutation into the polynucleotide sequence to exchange one nucleotide for another nucleotide optionally resulting in a mutation in the corresponding polypeptide sequence may be accomplished by site-directed mutagenesis using any of the methods known in the art. Such techniques are explained in the literature, for example: Ausubel et al., eds., Current Protocols in Molecular Biology, John Wiley & Sons, New York, N.Y. (2002).

Particularly useful is the procedure that utilizes a super coiled, double stranded DNA vector with the polynucleotide sequence of interest and two polynucleotide primers harboring the mutation of interest. The primers are complementary to opposite strands of the vector and are extended during a thermocycling reaction using, for example, Pfu DNA polymerase. On incorporation of the primers, a mutated plasmid containing nicks is generated. Subsequently, this plasmid is digested with DpnI, which is specific for methylated and hemimethylated DNA to digest the start plasmid without destroying the mutated plasmid (see Example 2.1).

Other procedures know in the art for creating, identifying and isolating mutants may also be used, such as, for example, gene shuffling or phage display techniques.

According to another aspect of the invention there are provided isolated polynucleotides (including genomic DNA, genomic RNA, cDNA and mRNA; double stranded as well as +ve and −ve strands), which encode the polypeptides of the invention.

The polynucleotides can be synthesized chemically, or isolated by one of several approaches known to the person skilled in the art such as polymerase chain reaction (PCR) or ligase chain reaction (LCR) or by cloning from a genomic or cDNA library.

Once isolated or synthesized, a variety of expression vector/host systems may be used to express proCPU encoded proteins. These include, but are not limited to microorganisms such as bacteria expressed with plasmids, cosmids or bacteriophage; yeasts transformed with expression vectors; insect cell systems transfected with baculovirus expression systems; plant cell systems transfected with plant virus expression systems, such as cauliflower mosaic virus; or mammalian cell systems (for example those transfected with adenoviral vectors); selection of the most appropriate system is a matter of choice.

Expression vectors usually include an origin of replication, a promoter, a translation initiation site, optionally a signal peptide, a polyadenylation site, and a transcription termination site. These vectors also usually contain one or more antibiotic resistance marker gene(s) for selection. As noted above, suitable expression vectors may be plasmids, cosmids or viruses such as phage or retroviruses. Examples of suitable retroviral vectors that could be used include: pLNCX2 (Clontech, BD Biosciences, Cat #631503), pVPac-Eco (Stratagene, Cat #217569) or pFB-neo (Statagene, Cat #217561). Examples of packaging cell lines that might be used with these vectors include: BD EcoPack2-293 (Clontech, BD Biosciences, Cat #631507), BOSC 23 (ATCC, CRL-11270), or Phoenix-Eco (Nolan lab, Stanford University). The coding sequence of the polypeptide is placed under the control of an appropriate promoter (i.e. HSV, CMV, TK, RSV, SV40 etc), control elements and transcription terminator so that the nucleic acid sequence encoding the polypeptide is transcribed into RNA in the host cell transformed or transfected by the expression vector construct. The coding sequence may or may not contain a signal peptide or leader sequence for secretion of the polypeptide out of the host cell. Preferred vectors will usually comprise at least one multiple cloning site. In certain embodiments there will be a cloning site or multiple cloning site situated between the promoter and the gene of interest. Such cloning sites can be used to create N-terminal fusion proteins by cloning a second nucleic acid sequence into the cloning site so that it is contiguous and in-frame with the gene of interest. In other embodiments there may be a cloning site or multiple cloning site situated immediately downstream of the gene of interest to facilitate the creation of C-terminal fusions in a similar fashion to that for N-terminal fusions described above, may be expressed in a variety of hosts such as bacteria, plant cells, insect cells, fungal cells and human and animal cells. Eukaryotic recombinant host cells are particularly suitable. Examples include yeast, mammalian cells including cell lines of human, bovine, porcine, monkey and rodent origin, and insect cells including Drosophila, army fallworm and silkworm derived cell lines. A variety of mammalian expression vector/host systems may be used to express the variant proCPU and CPU proteins of the present invention. Particular examples include those adapted for expression using a recombinant adenoviral, adeno-associated viral (AAV) or retroviral system. Vaccinia virus, cytomegalovirus, herpes simplex virus, and defective hepatitis B virus systems, amongst others may also be used. Particular cell lines derived from mammalian species which may be used and which are commercially available include, L cells L-M(TK-) (ATCC CCL 1.3), L cells L-M (ATCC CCL 1.2), HEK 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

Although it is preferred that mammalian expression systems are used for expression of the variant proCPU or CPU gene, it will be understood that other vector and host cell systems such as, bacterial, yeast, plant, fungal, insect are also possible.

The vectors containing the DNA coding for the CPU polypeptides of the invention can be introduced into host cells to express a polypeptide of the present invention via any one of a number of techniques, including calcium phosphate transformation, DEAE-dextran transformation, cationic lipid mediated lipofection, electroporation or infection. Performance of the invention is neither dependent on nor limited to any particular strain of host cell or vector; those suitable for use in the invention will be apparent to, and a matter of choice for, the person skilled in the art.

Host cells genetically modified to include a mutant CPU encoding nucleotide sequence may be cultured under conditions suitable for the expression and recovery of the encoded proteins from the cell culture. Such expressed proteins/polypeptides may be secreted into the culture medium or they may be contained intracellularly depending on the sequences used, i.e. whether or not suitable secretion signal sequences were present.

Expression and purification of the polypeptides of the invention can be easily performed using methods well known in the art (for example as described in Sambrook et al., ibid).

Thus, in another aspect, the invention provides for cells and cell lines transformed or transfected with the nucleic acids of the present invention. The transformed cells may, for example, be mammalian, bacterial, yeast or insect cells. According to a further aspect of the invention there is provided a host cell adapted to express a mutant CPU polypeptide of the present invention.

A plasmid comprising a nucleotide sequence encoding a CPU mutant of the present invention represents a further aspect of the invention.

Suitable expression systems can also be employed to create transgenic animals capable of expressing proCPU (see for example, U.S. Pat. No. 5,714,666).

According to a further aspect of the invention there is provided a transgenic, non-human animal whose cells comprise a nucleic acid encoding a mutant CPU with increased thermal stability relative to wild-type CPU, and regulatory control sequences capable of directing expression of the gene in said cells. In a preferred embodiment the transgenic animal is murine, ovine or bovine.

According to a further aspect of the invention there is provided a host cell adapted to express a mutant proCPU or CPU polypeptide of the invention from the nucleic acid sequence of the invention. Preferred host cells are mammalian such as CHO-K1 or Phoenix cells. Human cells are most preferred for expression purposes.

The polypeptides of the invention, or convenient fragments thereof that comprise the substituted amino acid, may be used to raise selective antibodies. Such antibodies have a number of uses, which will be evident to the molecular biologist or immunologist of ordinary skill. Such uses include, but are not limited to, monitoring protein expression, development of assays to measure activity, precipitation or purification of the protein and as a diagnostic tool to detect the amounts of the CPU proteins. Enzyme linked immunosorbant assays (ELISAs) are well known in the art and would be particularly suitable for detecting the polypeptides of the invention, or fragments thereof. Antibodies can be prepared using any suitable method. For example, purified polypeptide may be utilized to prepare specific antibodies.

Thus according to a further aspect of the invention there is provided an antibody capable of selectively binding to a mutant CPU of the invention. By selectively binding we mean to the exclusion of wild-type CPU and other CPU mutants that do not possess the particular epitope against which the antibody binds.

Antibodies can be prepared using any suitable method. For example, purified polypeptide may be utilized to prepare specific antibodies. The term “antibodies” is meant to include polyclonal antibodies, monoclonal antibodies, and the various types of antibody constructs such as for example F(ab′)₂, Fab and single chain Fv. Antibodies are defined to be specifically binding if they bind the allelic variant of CPU with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

The more stable CPU forms of the invention are more suited to the generation of crystal structures of proCPU or CPU, which will allow the 3-D structure of the enzyme to be deduced. Such information could then be used in the in silico design of compounds capable of modulating the proteolytic activity of the protein.

Thus, according to another aspect of the invention there is provided the use of a CPU mutant polypeptide of the present invention in the formation of crystals of said CPU mutant.

A representative method of how to grow such crystals is described in example 8.

The invention also provides for a method of producing a crystal structure of a CPU or proCPU mutant polypeptide of the present invention, comprising expressing the mutant polypeptide in a recombinant host cell, isolating the polypeptide and
complexing it with Fab fragment and subsequently purifying the complex.
activating the pro-form alone or in complex with a Fab fragment and isolating the resulting active form, alone or in complex with a Fab fragment.
using a Fab fragment directed against part or all of the 327-357 region, and
complexing this with proCPU, CPU and their mutants.

According to this aspect of the invention, a Fab fragment directed against all or part of the 327-357 region of CPU is provided for complexing with wild-type or mutant forms of proCPU or CPU to increase the stability of the CPU protein.

According to a further aspect of the invention there is provided use of a Fab fragment that binds to all or part of the 327-357 region of CPU (according to the position in SEQ ID NO: 2) to enhance the stability of CPU or proCPU.

According to a further aspect of the invention there is provided a method of enhancing the stability of CPU or proCPU comprising, complexing isolated CPU or proCPU with a Fab fragment directed against all or part of amino acids 327-357 of CPU (according to the position in SEQ ID NO: 2).

The methods of producing crystals for structure determination by X-ray crystallography include any standard techniques such as vapor diffusion, dialysis, batch crystallization and free interface diffusion. For further information the reader is referred to Protein crystallization: A laboratory manual, Bergfors (ed.) International University line 1999. To aid crystallization the protein can be stabilized by adding a ligand, for example (2-guadininoethylmercapto)succinic acid, or by coupling the enzyme to a monoclonal antibody. A representative method of how to grow such crystals is described in Example 8.

According to a further aspect of the invention there is provided a crystal of a mutant CPU polypeptide of the present invention, or a crystal of a proCPU mutant of the invention, or a crystal of CPU Fab fragment complex or a crystal of proCPU Fab fragment complex.

The more stable CPU mutant forms of the invention may also have therapeutic value, for example as an effective procoagulant biotherapeutic or as an antifibrinolytic biotherapeutic. The protein could be expressed via recombinant means to produce the CPU or proCPU polypeptide and formulated for systemic administration to patients in need of such an agent, for example in coagulation therapy.

Thus, according to a further aspect of the invention there is provided the use of a CPU mutant polypeptide according to the present invention in the manufacture of a medicament. In one embodiment the medicament is a procoagulant. In another embodiment the medicament is for treating, preventing, managing or ameliorating the symptoms of hemorrhagic disease or disorder. In certain embodiments the hemorrhagic disease or disorder includes, but is not limited to, hemophilia, von Willebrand disease (VWD), Henoch-Schonlein purpura and coagulation and fibrinolysis factor deficiencies.

The hemorrhagic diseases or disorders occur, in part, because the normal balance between the coagulation and fibrinolytic cascades has been affected, altered or shifted. The mutants of the present invention allow particular imbalances of the cascades to be corrected.

According to a further aspect of the invention there is provided the use of a mutant CPU polypeptide of the present invention in the treatment of a patient suffering from systemic bleeding.

According to a further aspect of the invention there is provided the use of a mutant CPU polypeptide of the present invention as an antidote to systemic bleeding caused by anti-coagulant therapy.

According to a further aspect of the invention there is provided a pharmaceutical composition comprising a therapeutically effective amount of a mutant CPU according to the present invention and a pharmaceutically effective excipient or diluent.

According to a further aspect of the invention there is provided a method of treating a hemorrhagic disease or disorder comprising administration of a therapeutically effective amount of a CPU mutant according to the present invention to a patient in need thereof.

According to a further aspect of the invention there is provided a method of prolonging fibrinolysis comprising contacting the blood with an effective amount of a CPU mutant of the present invention.

According to a further aspect of the invention there is provided a method of treating, preventing or managing bleeding side-effects associated with the administration of tissue-lasminogen activator (t-PA), or an analog thereof, or other anti-coagulants, comprising administering a therapeutically or prophylactically effective amount of a CPU mutant polypeptide according to the present invention, or a pharmaceutical composition thereof, to a patient in need thereof.

Protein-based therapeutics are usually stored frozen, refrigerated, at room temperature, and/or in a freeze-dried state.

The compositions of the invention may be obtained by conventional procedures using conventional pharmaceutical excipients, well known in the art, but will most likely be in a form suitable for injection, either parenterally or directly into the wound site.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Powders suitable for preparation of an aqueous preparation for injection, by the addition of a suitable diluent, generally contain the active ingredient together with suitable carriers and excipients, suspending agent and one or more stabilizers or preservatives. The diluent may contain other suitable excipients, such as preservatives, tonicity modifiers and stabilizers.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate.

The pharmaceutical compositions of the invention may also be in the form of a sterile solution or suspension in a non-toxic parenterally acceptable diluent or solvent, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

For further information on Formulation the reader is referred to Chapter 25.2 in Volume 5 of Comprehensive Medicinal Chemistry (Corwin Hansch; Chairman of Editorial Board), Pergamon Press 1990; or, Volume 99 of Drugs and the pharmaceutical sciences; Protein formulation and delivery (Eugen J. McNally, executive editor), Marcel Dekker Inc 2000.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used.

Unless defined otherwise, all technical and scientific terms used herein have the same meanings as commonly understood by one of ordinary skill in the art to which this invention belongs. All publications mentioned herein are incorporated herein by reference.

The invention will be further described by reference to the following non-limiting Examples and figures.

The practice of the present invention will employ, unless otherwise indicated, conventional methods of virology, immunology, microbiology, molecular biology and recombinant DNA techniques within the skill of the art. Such techniques are explained fully in the literature. See, e.g., Sambrook et al., eds., Molecular Cloning: A Laboratory Manual (3^{rd} ed.) Cold Spring Harbor Laboratory Press, Cold Spring Harbor, N.Y. (2001); Ausubel et al., eds., Current Protocols in Molecular Biology, John Wiley & Sons, New York, N.Y. (2002); Glover & Hames, eds., DNA Cloning 3: A Practical Approach, Vols. I, II, & III, IRL Press, Oxford (1995); Colowick & Kaplan, eds., Methods in Enzymology, Academic Press; Weir et al., eds., Handbook of Experimental Immunology, 5^{th} ed., Blackwell Scientific Publications, Ltd., Edinburgh, (1997); Fields, Knipe, & Howley, eds., Fields Virology (3^{rd} ed.) Vols. I & II, Lippincott Williams & Wilkins Pubs. (1996); Flint, et al., eds., Principles of Virology: Molecular Biology, Pathogenesis, and Control, ASM Press, (1999); Coligan et al., eds., Current Protocols in Immunology, John Wiley & Sons, New York, N.Y. (2002).

### EXAMPLE 1

### Cloning of Human PreproCPU cDNA and Subcloning into Various Vectors See Strömqvist et al., Clinica Chimica Acta. 347:49-59, 2004.

Total mRNA was isolated from human liver biopsies using oligo (dT) cellulose columns and total cDNA was synthesized with Superscript™ (Invitrogen, Cat No #18090). A 1.3 kb proCPU cDNA fragment was isolated using sequence-specific oligonucleotides (SEQ ID NO: 8 and SEQ ID NO: 9). The fragment was cloned into pUC18 (Fermentas, Cat ##SD0051) at SmaI site, the cDNA insert was sequenced on both strands and confirmed to encode human proCPU and designated as pAM48.

In order to generate an expression vector for production of recombinant proCPU in mammalian cells, two primers were synthesized: 1. reverse primer (SEQ ID NO: 10) containing the 3′part of the mouse metallothionein 1 (mMT-1) promoter region and the first 20 base-pairs, ATG and a HindIII-site of human proCPU cDNA. This oligonucleotide was used together with a primer; 2. Forward primer (SEQ ID NO: 11) that is complementary to a part of mMT-1 promoter, in a PCR-reaction. The PCR-product was amplified using AmpliTaq® (Perkin Elmer Cetus Instr.) and cloned into a pCR™ Vector (Invitrogen), for sequence analysis. This plasmid was digested with SacI-HindIII and a fragment of 239 bp (containing mMT-1 promoter 3′ and the first 20 bp of pro-CPU 5′) and ligated with a 444-bp HindIII-BamHI fragment (containing proCPU 5′) from the plasmid pAM48. These two fragments were subcloned into SacI- and BamHI-digested pUC19 (Fermentas, Cat ##SD0061). The resulting clone was designated pAM215. In order to facilitate further cloning of the expression vector, pAM215 was first digested with SacI and BamHI and a 683 bp fragment was isolated. Second, the vector pS147 (Hansson et al. J. Biol. Chem. 268: 26692-26698, 1993) was digested with SacI and SalI, and a fragment of 12.9 kb was isolated. This fragment contains the distal part of the murine metallothionein-1 (mMT-1) upstream regulatory element (Pavlakis and Hamer (Proc. Natl. Acad. Sci. U.S.A. 80:397-401, 1983)) the bovine papilloma virus sequences, the rabbit β-globin genomic fragment providing mRNA processing signals and the plasmid sequences, pML2d (Sarver et al. Proc. Natl. Acad. Sci. U.S.A. 79:7147-7151, 1982). Third, to isolate the 3′part of human proCPU, the plasmid pAM82 (pAM82 contains a proCPU cDNA NdeI-SacI fragment from pAM48 in pET28a(+) HisTag, Novagen, Cat #69864-3) was digested with BamHI and SalI and an 898-bp fragment was isolated. The ligation of these three fragments resulted in the expression vector pAM227.

To create the plasmid pAM245, the proCPU cDNA was subcloned as a BglII-SalI fragment from plasmid pAM227, into the BamHI-SalI sites of the pFAST-Bac1 (Invitrogen, Cat #10360-014) baculovirus transfer vector.

### EXAMPLE 2

### Generation and Discovery of CPU Mutants With Increased Thermostability

The following two examples show how random and directed nucleotide substitutions were introduced into the preproCPU cDNA sequence. It also shows how these mutations were further combined and CPU variants with increased thermostability identified from a large number of mutants.

2.1. Site-Directed Mutagenesis of the PreproCPU cDNA

Directed nucleotide substitutions were introduced into the preproCPU cDNA with the Quikchange XL site-directed mutagenesis kit (Stratagene, Cat #200516) according to the manufacturer's instructions.

Site-mutagenesis could also be performed using other techniques known in the art. Such techniques are explained in the literature, for example: Ausubel et al., eds., Current Protocols in Molecular Biology, John Wiley & Sons, New York, N.Y. (2002).

2.2. Random Mutagenesis of the PreproCPU cDNA

Error-prone PCR was performed according to Cadwell and Joyce (PCR Methods Appl. 2(1):28-33, 1992 and 3(6):S136-140, 1994). The 100 μL reaction mixture contained 1 fmole of preproCPU cDNA (SEQ ID NO: 1) in one of the vectors described in example 1, 50 mM KCl, 10 mM Tris.HCl pH 8.3, 7 mM MgCl₂, 0.01% (weight/volume) gelatin, 0.3 μM of each primer CPU_fwd_XhoI (SEQ ID NO: 14) and CPU_rev_NotI (SEQ ID NO: 15), 0.2 mM DATP, 0.2 mM dGTP, 1 mM dTTP, 1 mM dCTP, 0.5 mM MnCl₂, and 2.5 U of AmpliTaq DNA polymerase (Applied Biosystems, Cat #N8080171). The cycling parameters used were: 94° C. for 2 min, followed by 30 cycles, each consisting of denaturation at 94° C. for 30 s, annealing at 45° C. for 45 s, and elongation at 72° C. for 1 min, followed by 72° C. for 7 min.

The second type of random mutagenesis was performed with the Genemorph PCR mutagenesis kit (Stratagene, Cat #600550) according to the manufacturer's instructions. The mutant preproCPU cDNAs were ligated into the multiple cloning site of a retroviral vector, for functionality evaluation (see below), or subcloned for sequencing using the pGEM-T vector system II (Promega, Cat #A3610) according to the manufacturer's instructions.

2.3. Random Recombination of Mutated PreproCPU cDNAs

Random recombination of preproCPU cDNAs (mutated as described above) was performed using in vitro molecular evolution of protein function procedure (now known as Fragment-INduced Diversity (FIND) technology) according to the methods disclosed in UK Patent Publication No. GB 2 370 038A, wherein single stranded template DNA is made using, for example, M13 phage, the ±ve and −ve single strands are separately digested with nucleases such as Bal31, the two treated single stranded DNA pools are mixed and the gene is reassembled in a shuffled nature by the use of two subsequent PCR reactions. This essentially fragments the mutation containing nucleic acid and recombines them to generate nucleic acids that possess combinations of the original mutations.

### 2.4. Generation of Stable Cell Lines Expressing ProCPU and Mutant ProCPUs

PreproCPU mutant proteins can be prepared by expressing the mutated preproCPU cDNAs in any conventional expression system. A retroviral gene delivery and expression system was used by the present inventors.

DNA of the mutant preproCPU cDNAs in the retroviral vector (see above) were transformed into XL1-Blue electroporation-competent cells (Stratagene, Cat #200228) according to the manufacturer's instructions. The resulting colonies were cultured (3 h, 37° C., 220 rpm) for subsequent plasmid purification to yield mutant preproCPU library DNA.

3T3 cells (ATCC, Cat #CRL-1658) and a MMLV-based packaging cell line suitable for use with the retroviral vector (Miller (1997). Development and Applications of Retroviral Vectors. In Retroviruses, J. M. Coffin, S. H. Hughes and H. E. Varmus (Eds.), pp. 437-473. Cold Spring Harbor Laboratory Press, Plainview, N.Y.) were cultured (37° C., 5% CO₂) in D5% (Dulbecco's modified Eagle's medium (Sigma, Cat #D5796) supplemented with 5% fetal bovine serum (HyClone, Cat #SH30084), heat inactivated at 63° C. for 30 min, and 1% nonessential amino acids (Invitrogen, Cat #11140)).

Stable cell lines were generated as described by Krebs et al. (Methods Cell Sci. 21:57-68, 1999). Briefly, 2.5 μg of the mutant library DNA was transiently transfected into the packaging cell line (80-90% confluent, 10 cm² culture plate, 2 mL D5%) using Lipofectamine 2000 (Invitrogen, Cat #11668) according to the manufacturer's instructions. The medium was replaced with D5% 5 h post transfection and 48 h later the virus containing supernatants were collected and passed through 0.45 μm filters. The supernatants (400 μL), together with polybrene (final concentration 10 μg/mL, Sigma, Cat #H9268), were added to the 3T3 cell line (80-90% confluent, 10 cm² culture plate, 2 mL D5%). The medium was replaced 16 h post infection with D5%+G418 (D5% supplemented with 0.8 mg/mL G418 (Invitrogen, Cat #11811)) in order to select for stable transfectants. Following 4-5 days of selection, the cells were cultured individually in 150 μL D5%+G418 in 96-well plates for 19 days without splitting before expressed proCPU was analysed for stability (see below). Selected clones were regrown and analyzed after culturing for 10-12 days in 24-well plates without splitting.

Stable transfectants expressing site-directed mutated proCPU were generated as described above and cultured in D5%+G418 for at least two weeks before analysis of proCPU stability (see below).

### 2.5. Screening for Improved CPU Stability

The following example outlines how over 5000 proCPU clones were screened for improved thermostability of the active CPU form after DNA recombination using FIND technology of selected clones from a randomly mutated library.
1. 10 μL supernatant from the cultivation plates was transferred to 384 well microtiter plates using a Multimek pipetting robot (Beckman Coulter).
2. The activity of CPU was determined according to the method (Hippuricase assay) described by Schatteman et al. (Clin Chem Lab Med. 39(9): 806-810, 2001). In the first step the proCPU was activated by addition of 5 μL (24 nM thrombin from human plasma, Sigma-Aldrich, Cat #T-8885, and 48 nM thrombomodulin from rabbit lung, American Diagnostica, Cat #237, in 20 mM Hepes pH 7.4 containing 5 mM CaCl₂) and incubated at room temperature for 10 min.
3. The activation was stopped by addition of 5 μL 20 μM phenylalanyl-prolyl-arginyl-chloromethyl ketone (Calbiochem, Cat #520222) in 20 mM Hepes pH 7.4 containing 5 mM CaCl₂.
4. Thermal stability was assessed by incubating activated CPU at 37° C. for 90 min.
5. The remaining CPU activity was determined by addition of 30 μL substrate solution (8 mM p-Hydroxyhippuryl-Arg-OH (Bachem, Cat #G-3610), 2.5 mM 4-Aminoantipyrine (Merck, Cat #107293) and 2 U/mL Hippuricase (EC 3.5.1.14, purified as described in Schatteman et al. (Clin Chem Lab Med. 39(9): 806-810, 2001) in 100 mM Hepes pH 7.6 and incubated at 37° C. for 1 h.
6. The reaction was stopped by addition of 30 μl stop solution (12 mM NaIO₄ and 35 mM EDTA) and incubated at 37° C. for 20 min. The absorbance at 505 nm (A⁹⁰) was measured in a Polarstar plate reader from BMG (Germany).

Since the A⁹⁰ of the heat-inactivated clones in the primary screening also depends, to some extent, on both activity and expression level, the resulting absorbance value is an indication of stability. About 8% of the clones showed significantly higher A⁹⁰ than the best parental clone used in the DNA recombination step. 380 clones expressing highest A⁹⁰ were picked and transferred to two new 384-well microtiter plates.

A secondary screening of these 380 clones was used to verify the hits in the primary screening and to correct for variations in expression level. In one plate the initial CPU activity (A⁰) before heat inactivation was determined using the same protocol as in the primary screen but without any inactivation and in the second plate the A⁹⁰ was assessed using the same protocol as in the primary screen. A stability index was determined for each clone as the quote (A⁹⁰/A⁰) to exclude any influence of different expression level and activity. Approximately 50 clones with improved stability index were selected and re-grown.

The stability of the re-grown clones was determined by incubating the activated CPU at 37° C., and periodically withdrawing sample and assaying for activity using the same protocol as used in the primary screening described above. The A⁰ values were plotted vs. time and the apparent half-life (T½) was calculated by fitting the data to an exponential decay function (Y=Span*exp(−K*X)+Plateau) using software Prism 3.0 (GraphPad).

The relative specific activity versus wild type CPU of the re-grown clones was determined by measuring the initial activity and the proCPU concentration using a proCPU ELISA as described by Strömqvist et al. (Thromb Haemost. 85: 12-17, 2001). To determine the initial activity four reactions were run on each clone and stopped after 5, 10, 15 and 20 min and the resulting slope when plotting absorbance at 492 versus time was used as initial activity.

### 2.6. Determination of the ORF of ProCPU Stably Expressed in 3T3 Cells

After analysis of secreted proCPU (see above), RNA was purified from selected stable 3T3 cell lines using Trizol (Invitrogen, Cat #15596) according to the manufacturer's instructions. Reverse transcription-PCR using CPU_fwd_XhoI and CPU_rev_NotI as primers (see above) was performed with the Titan RT-PCR kit (Roche, Cat #1939823) according to the manufacturer's instructions. The PCR products were subcloned into pGEM-T for sequencing.

TABLE 2
Half-life (T½) of different CPU mutants at 37° C. created by site
directed or random mutagenesis. The remaining enzymatic activity after
incubation of CPU or its mutants at 37° C. was determined either using
a HPLC assay (see example 6) or the Hippuricase assay (see example 2).
The table also shows all mutations found in the ORF of preproCPU at the
amino acid level for each clone (for details of expression and selection of
the clones see example 2).

| | Amino acid change | T½ at 37° C. | T½ at 37° C. |
|---|---|---|---|
| | with respect to SEQ | (min) | (min) |
| Clone | ID NO: 2 | Hippuricase assay | HPLC assay |
| EP4:44B7 | K166N, H357Q | 40 | 31 |
| EP4:18G3 | I251T, H357P | 35 | 39, 22 |
| GM2:65D2 | H315R, S327C | 27 | 60 |
| GM2:7E3 | H355Y | 18 | 47 |
| EP4:50F10 | I180F*, H357Q | 50 | 61, 49 |
| S11 | L376Q | 12.4 | 16.1 |
| ST | T347I | 10.1 | 17.8 |
| WT | — | 7.8 | 12 |

*this mutation was not present in all PCR products derived from this clone

For the first round of FIND approach (see 2.3.) the following clones from Table 2 were used: EP4:44B7, EP4:18G3, GM2:65D2, GM2:7E3, EP4:50F10, S11, ST.

Libraries created from these clones by FIND technology were expressed, screened and characterized as described in example 2. In parallel, the clones HQ and HP were constructed from GM2:7E3 (table 1) by site directed mutagenesis (see 2.1). Table 3 summarizes clones derived from this step.

TABLE 3
Half-life (T½) of different CPU mutants at 37° C. derived from the 1^{st}
round of FIND treatment and site directed mutagenesis. The enzymatic activity
remaining after incubation of CPU or its mutants at 37° C. was determined
using either a HPLC assay (see example 6) or the Hippuricase assay (see above).
If more than two determinations were made, the T½ is reported as mean ± SD
and the number of determinations is indicated (n). The table also shows all mutations
found in the ORF of preproCPU at the nucleotide and amino acid level for each clone.

| | | | Nucleotide | Amino acid |
|---|---|---|---|---|
| | T½ at 37° C. | T½ at 37° C. (h) | change with | change with |
| | (h) | Hippuricase | respect to SEQ | respect to SEQ ID |
| Clone | HPLC assay | assay | ID NO: 1 | NO: 2 |
| F1.1.11E3 | 2.2 | >1 | T752C | I251T |
| | | | A894G | |
| | | | A944G | H315R |
| | | | A979T | S327C |
| | | | A1049G | N350S |
| | | | T1071A | H357Q |
| F1.1.21B10 | >1 | >1 | A375G | |
| | | | A498T | K166N |
| | | | T534C | |
| | | | A944G | H315R |
| | | | A979T | S327C |
| | | | A1049G | N350S |
| | | | T1071A | H357Q |
| F1.1.65B7 | >1 | >1 | A375G | |
| | | | A498T | K166N |
| | | | T534C | |
| | | | G693A | |
| | | | A944G | H315R |
| | | | A979T | S327C |
| | | | A1070C | H357P |
| F1.1.65C3 | 1.6 | >1 | G693A | |
| | | | A944G | H315R |
| | | | A979T | S327C |
| | | | G1055A | R352K |
| F1.1.65E2 | >1 | >1 | A944G | H315R |
| | | | A979T | S327C |
| | | | A1049G | N350S |
| | | | T1071A | H357Q |
| F1.1.71F5 | >1 | >1 | C357T | |
| | | | A894G | |
| | | | A979T | S327C |
| | | | G1043A | S348N |
| | | | T1071A | H357Q |
| F1.2.28G7 | 2.2 | not done | A944G | H315R |
| | | | A979T | S327C |
| | | | C1063T | H355Y |
| F1.2.44B9 | 1 | not done | T656C | V219A |
| | | | A944G | H315R |
| | | | A979T | S327C |
| HP | 1.5 | not done | C1063T | H355Y |
| | | | A1070C | H357P |
| HQ | >1 | >1 | C1063T | H355Y |
| | | | T1071A | H357Q |
| Wild-type | 0.2 ± 0.03 | 0.13 ± 0.02 | — | — |
| | (n = 3) | (n = 21) | | |

After finishing the 1^{st} round of FIND treatment new mutants were made by site directed mutagenesis (see 2.1) from some of the clones found in the first round of FIND treatment and the random libraries (Table 2). They were expressed and characterized as described in the examples 2 and 3. They are summarized in Table 4.

TABLE 4
Half-life (T½) of different CPU mutants at 37° C. made from
clones in Table 3 and 2. The remaining enzymatic activity after incubation
of CPU or its mutants at 37° C. was determined either using a HPLC
assay (see example 6) or the Hippuricase assay (see above).
The table also shows all mutations found in the ORF of preproCPU at the
amino acid level for each clone (for details of expression and selection
of the clones see example 2).

| | Amino acid | | |
|---|---|---|---|
| | change with | T½ at 37° C. (h) | T½ at |
| | respect to SEQ ID | Hippuricase | 37° C. (h) |
| Clone | NO: 2 | assay | HPLC assay |
| GM2.7E3 + T347I* | T347I, H355Y | Not done | Not done |
| F1.1.65B7 + R315H | K166N, S327C, | >1 | >1 |
| | H357P | | |
| F1.1.71F5 + S327P | S327P, S348N, | 0.3 | Not done |
| | H357Q | | |
| F1.1.11E3 + R315H | I251T, S327C, | >1 | 0.7 |
| | N350S, H357Q | | |
| F1.2.28G7 + R315H | S327C, H355Y | Not done | >1 |
| F1.1.71F5 + N348S | S327C, H357Q | Not done | >1 |
| F1.1.71F5 + H355Y | S327C, S348N, | Not done | >1 |
| | H355Y, H357Q | | |
| HQ + S348N | S348N, H355Y, | >1 | >1 |
| | H357Q | | |
| HQ + T347I | T347I, H355Y, | >1 | >1 |
| | H357Q | | |
| HQ + S327P | S327P, H355Y, | 1.0 | 1.1 |
| | H357Q | | |
| HQ + N350S | N350S, H355Y, | >1 | >1 |
| | H357Q | | |
| WT | — | 0.13 | 0.2 |

*very low activity did not allow T½ determinations for GM2.7E3 + T347I

Then for a second round of FIND treatment the clones: GM2.7E3+T347I, F1.1.65B7+R315H, F1.1.71F5+S327P, F1.1.11E3 and HQ (see Table 3 and 4) were used. Libraries created from these clones by FIND technology were expressed, screened and characterized as described in example 2. Table 5 summarizes clones derived from this approach.

TABLE 5
Half-life (T½) of different CPU mutants at 37° C. derived from the
2^{nd} round of FIND treatment. The remaining enzymatic activity
after incubation of CPU or its mutants at 37° C. was determined either
using a HPLC assay (see example 6) or the Hippuricase assay (see
above). The table also shows all mutations found in the ORF of
preproCPU at the amino acid level for each clone (for details of
expression and selection of the clones see example 2).

| | Amino acid change | | T½ at 37° C. |
|---|---|---|---|
| | with respect to SEQ | T½ at 37° C. (h) | (h) |
| Clone | ID NO: 2 | Hippuricase assay | HPLC assay |
| F2.1-31F7 | I251T, H355Y, | >1 | >1 |
| | H357Q | | |
| F2.1-47C11 | , I204T, Y230C, | >1 | >1 |
| | S348N, H357Q | | |
| F2.1-60G8 | S327C, H355Y, | >1 | >1 |
| | H357Q | | |
| WT | — | 0.13 | 0.2 |

The following two clones were also made and characterized:

TABLE 6
Half-life (T½) of different CPU mutants at 37° C. The remaining
enzymatic activity after incubation of CPU or its mutants at 37° C.
was determined either using a HPLC assay (see example 6) or the
Hippuricase assay (see above). The table also shows all mutations
found in the ORF of preproCPU at the amino acid level for each
clone (for details of expression and selection of the clones see
example 2).

| | | T½ at | T½ at |
|---|---|---|---|
| | Amino acid change with | 37° C. (h) | 37° C. (h) |
| Clone | respect to SEQ ID NO: 2 | Hippuricase assay | HPLC assay |
| F2.239C3 | K166N, S327C, | >1 | Not done |
| | K349R*, H355Y, | | |
| | H357Q | | |
| F2.2.134E11 | S327C, K346N, H355Y, | >1 | Not done |
| | H357Q | | |
| WT | — | 0.13 | 0.2 |

*this mutation was not present in all PCR products derived from this clone

### EXAMPLE 3

### Expression of ProCPU in Insect Cells and its Purification from the Supernatant of Infected Insect Cells

The following example shows how proCPU (or a mutant proCPU) can be expressed in insect cells as a C-terminal octa His tagged protein. It also shows how proCPU (or mutant proCPU) with a C-terminal His-tag can be purified from the supernatant of infected SF9 insect cells by IMAC.

### 3.1. Expression of ProCPU in Insect Cells

The ORF of preproCPU (SEQ ID NO: 1) was amplified in a PCR reaction using pAM245 (described in example 1) as the template and the following primers:
Forward: CPU-for1 (SEQ ID NO: 3)
Reverse: C-HIS1rev (SEQ ID NO: 4) and C-HIS2rev (SEQ ID NO: 5)

The resulting PCR fragment was digested with NotI/KpnI and ligated into the NotI/KpnI sites of pFAST-Bac1 (Invitrogen, Cat #10360-014). The primers C-HIS1 rev (SEQ ID NO: 4) and C-HIS2rev (SEQ ID NO: 5) introduced the coding sequence for an octa-His tag at the C-terminus of proCPU (amino acid sequence of the tag: LEPGDDDDKHHHHHHHHSGS—SEQ ID NO: 16). The resulting plasmid was named pAM1079.

Recombinant baculovirus for expression of recombinant proCPU with C-terminal octa-His tag (proCPU-CHis) was generated starting from pAM1079 with the Bac-to-Bac® Baculovirus Expression System (Invitrogen, Cat #10359-016) according to the manufacturer's instructions. Recombinant proCPU-CHis expression was detected by the proCPU ELISA described by Strömqvist et al. (Thromb Haemost. 85: 12-17, 2001).

### 3.2. Purification of ProCPU

SF9 insect cells (Invitrogen, Cat #11496-015) were kept in shaker culture (27° C., 105 rpm) in Sf-900II SFM medium (Invitrogen, Cat #10902-088) and were infected at a MOI>1. The supernatant was harvested after 3 to 5 days by centrifugation for 45 min at 6.000×g. The supernatant was subsequently concentrated approximately 4-times using vivaflow 50 units with a MWCO 10.000 (Vivascience, Cat #VF05CO). The concentrated supernatant was dialysed overnight against 50 mM NaH₂PO₄, 300 mM NaCl pH 7 (buffer A). The dialysed supernatant was loaded on a Talon™Superflow™ (Clontech, Cat #8908-1) column. The column was first washed with 5 column volumes buffer, then with a gradient up to 45 mM imidazole in buffer A (5 column volumes) followed by 5 column volumes of 45 mM imidazole in buffer A. Elution of proCPU-CHis was done by a linear gradient (2 column volumes) from 45 to 125 mM imidazole in buffer A.

ProCPU-CHis containing fractions were pooled and buffer exchange into 20 mM Hepes, 150 mM NaCl pH 7.4 was performed using PD10 columns (Amersham Biosciences, Cat #17-0851-01) according to the manufacturer's instructions.

### 3.3. Expression and Purification of Mutant ProCPUs

The ORF of the mutant preproCPUs (here designated in general as mutant X) were amplified by PCR from the plasmids described in example 2 using the following primers:
Forward: GateCPUfor (SEQ ID NO: 6).
Reverse: C-HIS1 rev (SEQ ID NO: 4) and C-HIS2rev (SEQ ID NO: 5) and GateHISrev (SEQ ID NO: 7).

The resulting PCR fragments were subcloned into the entry vector pDONR201 (Invitrogen, Cat #11798-014) using the Gateway™ Technology with help of a BP reaction (Invitrogen, Cat #11789-013) according to the manufacturer's instructions. The resulting plasmids were named pDONR201-mutant X proCPU-CHis.

Additional site directed mutagenesis on the inserts within these plasmids, if desired, was performed as described in example 2.1

Recombinant baculovirus for expression of recombinant mutant proCPU with C-terminal octa-His tag (mutant X proCPU-CHis) was generated starting from pDONR201-mutant X proCPU-CHis with the BaculoDirect™ Baculovirus Expression System (Invitrogen, Cat #12562-013 and 12562-039) according to the manufacturer's instructions.

Alternatively, recombinant baculovirus for expression of recombinant mutant proCPU with C-terminal octa-His tag (mutant X proCPU-CHis) was generated starting from pDONR201-mutant X proCPU-CHis with the Bac-to-Bac® Baculovirus Expression System (Invitrogen, Cat #10359-016) according to the manufacturer's instructions. For this, pDEST8 (Invitrogen, Cat #11804-010) was used as the destination vector and the ORF of the mutant was transferred into pDEST8 with the help of a LR reaction (Invitrogen, Cat #11791-019) according to the manufacturer's instructions.

Recombinant mutant X proCPU-CHis expression was detected by the proCPU ELISA described by Strömqvist et al. (Thromb Haemost. 85: 12-17, 2001).

The purification of mutant proCPU can be done as described for proCPU-CHis before.

### EXAMPLE 4

### Purification of a ProCPU-Fab Complex

This example describes how proCPU can be bound to an anti-proCPU Fab fragment (for generation of anti-proCPU Fab fragments see example 7) and how the complex of both can be isolated.

Purified proCPU and Fab fragment were mixed in a 1:3 ratio (weight:weight) and incubated overnight at 4° C. to form complexes. Un-complexed proCPU and Fab were separated from the proCPU-Fab complex by gel-filtration chromatography. Briefly, a superdex™ 200 16/60 column (Amersham Biosciences, Cat #17-1069-01) was equilibrated in 10 mM Bicin, 150 mM NaCl, 5 mM CaCl₂ pH 8.5 (buffer B) and 3 mL proCPU-Fab mixture were loaded onto the column. The column was developed in buffer B and fractions containing the proCPU-Fab complex were pooled.

### EXAMPLE 5

### Purification of CPU

This example describes how CPU can be isolated after cleavage of the pro-peptide of proCPU.

A way to activate proCPU to CPU is described in example 6. CPU is separated from un-activated proCPU and the pro-peptide by gel-filtration chromatography. Briefly, a superdex™ 75 16/60 column (Amersham Biosciences, Cat #17-1068-01) was equilibrated in 10 mM Bicin, 150 mM NaCl, 13 mM n-Octyl β-d Glykopyranosid pH 8.5 (buffer C) and the activated sample was loaded onto the column. The column was developed in buffer C and fractions containing the CPU complex were pooled.

An alternative way to isolate CPU is described in Mao et al. (Analytical Biochemistry. 319:159-170, 2003).

### EXAMPLE 6

### Stabilization of CPU by Anti-ProCPU Fab Fragments

This example shows how CPU activity can be measured by a HPLC based activity assay. It also teaches how proCPU can be activated to CPU and demonstrates that incubation of CPU with anti-proCPU Fab fragments (for generation of anti-proCPU Fab fragments see example 7) increases the half-live of CPU.

### 6.1. Reagents

The CPU substrate, hippuryl-arginine (Hip-Arg) was purchased from Sigma (St Louise, USA; Cat #H-2508) and dissolved in 50 mmol/L Hepes buffer to a final concentration of 30 mmol/L. Aliquots of the stock solution were stored at −20° C., then thawed and sonicated prior to use. Thrombin was obtained from Sigma (St Louis, Mo., USA; T-8885). One vial containing 10 U was dissolved in 1 mL 5 mmol/L CaCl₂ to yield a stock solution of 190 nmol/L. Rabbit lung thrombomodulin (TM), 30 U per vial, was purchased from American Diagnostics (Greenwich, Conn., USA; Cat #237) and dissolved to a stock solution of 430 nmol/L. Internal standard (IS), 2-Methylhippuric acid, from Aldrich (Steinheim, Germany; Cat #32800-6) dissolved in 25 mL 99.5% EtOH and made up to 100 mL with distilled H₂O. Aliquots were stored at −20° C. The solution was sonicated briefly prior to use and the internal standard was diluted 3-4 times with 25% EtOH before addition to the assay.

### 6.2. HPLC Analyzing System

The HPLC analyzing system employed consisted of an ASI 100 Automated Sampler Injector (Dionex Corp Sunnywale, Calif.) equipped with a high precision pump, model 480, UV-detector UVD 170U and a degasser, Degasys Populaire DP2003. All items were purchased from Gynkotek (Munchen, Germany). The mobile phase for analysis of CPU was for the 50*4.6 mm Econosphere™ C18 3u columns as follows: isocratic elution, 90% KH₂PO₄ 10 mmol/L, (adjusted to pH 3.5 with 10% H₃PO₄), and 10% acetonitrile. The flow rate was set to 1 mL/min and the elution time was 2 to 3 min for the 50 mm column. The software used was Chromeleon™, version 6.4 and the parameters analyzed from the HPLC chromatogram was the area under the curve (AUC) for the hippuric acid peak which was generated by CPU in the unknown sample, and AUC for the internal standard peak (IS). The AUC-values for the unknown sample are then divided with AUC for corresponding IS peaks to give the ratio: AUC_{unknown sample}/AUC_{IS}.

### 6.3. Activation of ProCPU

Activation of proCPU was performed as follows: 100 μL thrombin (12 nmol/L) was mixed with 100 μL thrombomodulin (48 nmol/L) and 100 μL proCPU, and incubated for 10 minutes at room temperature. Thrombin was then inhibited by the addition of 100 μL of the irreversible thrombin inhibitor PPACK (Alexis Cat #260-001-005) to a final concentration of 5 μmol/L.

### 6.4. Principle of HPLC Assay

The basic carboxypeptidase CPU acts on the substrate hippuryl-arginine (Hip-Arg). When arginine (Arg) is cleaved from the C-terminal portion of the substrate, the product hippuric acid is formed. Hippuric acid finally, is detected and quantified by means of High Pressure Liquid Chromatography (HPLC).

### 6.5. HPLC Assay

Each single assay was always analyzed together with an internal standard to which the product peak, generated by the unknown sample, is correlated (see below). The assay procedure was as follows: 40 μL of the substrate hippuryl-arginine (30 mmol/L) was first added to the vials. The assay was then started by the addition of 5 μL of the sample to be tested with 10 seconds interval. The vials were then incubated at 37° C. for 30 minutes after which the reactions were stopped by the addition of 50 μL HCl 1 mol/L in the same order and with the same interval (10 sec) as they were started. Ten μL of the internal standard (2-methylhippuric acid) and 300 μL ethyl acetate were then added to all vials and mixed properly by tilting the vials upside down 30 times before centrifuging for 1 minute at 1000*g. Two hundred μL from the upper phase (ethyl acetate) was then carefully collected and transferred into HPLC-vials and evaporated to dryness under N₂. Finally, the evaporated samples were dissolved in 75 μL mobile phase and 25 μL was analyzed in an HPLC-analyzing system.

### 6.7. Determination of Half-Life (T½) of CPU Alone or in Complex to a Fab Fragment

The aim was to measure T½ at 37° C. for CPU alone and CPU+ an anti-proCPU Fab fragment). The assay concentration of CPU was 0.15 μg/mL and the Fab fragments 7.5 μg/mL. After mixing CPU with Fab fragments the vials were pre-incubated at room temperature for 10 min. A start value was taken (0-value) before the vials were placed in a heatblock. Aliquots of 5 μL were then pipetted from each vial at the times 5, 10, 15, 20, 30, 45 and 60 min and the HPLC assay was immediately started.

### 6.8. Calculation of T½

The quota for each sample (HA/IS) y was plotted against corresponding time x and the T½ was then determined by first fitting equation 503 in Excel Fit (y=C+A*exp(−B*x)) to the data and then calculating T½ from B (T½=ln 2/B).

### Results:

TABLE 7
T½ of CPU alone or in complex to an antibody (or Fab fragment)
The T½ for CPU alone was arbitrarily set to 100%

| CPU+ | T½ (%) |
|---|---|
| alone | 100 |
| Anti-proCPU FAB747.86 | 126 |
| Anti-proCPU FAB752.13 | 125 |

### EXAMPLE 7

### Isolation of Fabs to ProCPU

This example shows how anti-proCPU Fab fragments can be generated, expressed and isolated.

Fab fragments were isolated from a naïve phage-display antibody library (Dyax Corporation) by three rounds of selection. This library comprises a wide range of different Fabs, each individually fused to a truncated version of bacteriophage p3 protein. (de Haard et al. Journal of Biological Chemistry. 274:18218-18230, 1999).

For each round of selection proCPU was passively absorbed to a 4 mL Immunotube™ (Nunc) overnight at 4° C. The tube was then emptied, washed with Dulbecco. A phosphate buffered saline; blocked by filling with a 2% solution of Marvel (Premier International Foods (UK) Ltd) in Dulbecco A phosphate buffered saline for 1 hour at room temperature. Phage, bearing a large naïve library of Fabs, on their surface were allowed to bind for 2 hours at room temperature. The tube was then extensively washed with Dulbecco A phosphate buffered saline with 0.1% Tween and Dulbecco A phosphate buffered saline alone. Thus removing non-specific phage-Fab. Phage, which remained bound, were eluted with 1 mL of 100 mM solution of triethanolamine for 10 minutes. This was immediately neutralized with 500 μL of 1M TRIS ph 7.4, then used to transfect a fresh population of E. coli strain TG1. This transfected population of E.coli was used to prepare a new batch of bacteriophage, which was used in the subsequent round of selection.

In the first round of selection the Immunotube™ was coated with 2 mL of proCPU @ 100 μg/ml in 0.1M carbonate buffer, in the second round with 2 mL of proCPU @ 30 μg/mL in 0.1M carbonate buffer, in the third round with 2 mL proCPU @ 10 μg/mL in 0.1M carbonate buffer

After three rounds of selection the eluted phage were used to transfect E.coli strain HB2151 (rather than strain TG1). Thus allowing the production of soluble Fab. These were plated for single colonies. 1000 colonies were picked into microtitre plates and grown. Expression was induced and periplasmic preparations made. These periplasmic preparations were tested by EIA against proCPU.

Positive individual colonies were “fingerprinted”. Briefly, PCR was used to amplify across the region encoding the Fab, and the PCR product was then cut with restriction enzyme (BstN1). The products were then separated on a 3% agarose gel. The patterns produced by individual colonies were examined for differences. The unique positive clones thus identified were expanded, and used to produce soluble Fab. Soluble Fab was purified by metal chelate chromatography using NiNTA (Qiagen).

### EXAMPLE 8

### Crystallization of (Pro)CPU

This example shows how for example proCPU or proCPU Fab fragment complexes can be crystallized.

Samples of proCPU or proCPU bound to anti-proCPU Fab fragments were concentrated in 20 mM Hepes pH 7.4, 150 mM NaCl to about 6 mg/mL using Millipore Ultrafree 0.5 centrifugal filter with a 10 kDa cut-off. In some cases samples were incubated with 1 mM (2-guadininoethylmercapto)succinic acid (purchased from Fluka) for about an hour. Crystallization trials were performed using the free interface diffusion technology (Hansen et al. Proceedings of the National Academy of Sciences of the United States of America. 99(26):16531-6, 2002). (Topaz™ Crystallizer, Fluidigm Corporation, 7100 Shoreline Court South San Francisco, Calif. 94080): 3 μL protein were used for a screening 48 different crystallization conditions from a sparse matrix screen, Fluidigm Microfluidics Crystallization Test Kit (Hampton Research). Crystals of proCPU alone or in complex with anti-proCPU Fab grew within 24 hours in several conditions.

### EXAMPLE 9

### Identities and Similarities of Mouse, Rat and Human PreproCPU

Sequence comparison was done with Blastp (Tatiana et al., “Blast 2 sequences—a new tool for comparing protein and nucleotide sequences”, FEMS Microbiol Lett. 174:247-250, 1999) and available at the NCBI homepage (http://www.ncbi.nlm.nih.gov/blast/b12seq/b12.html) using the default settings.

| | Mouse (SEQ ID NO: 12) | Rat (SEQ ID NO: 13) |
|---|---|---|
| Human | Identities = 349/422 | Identities = 345/422 |
| (SEQ ID NO: 2) | (82%), Positives = 378/ | (81%), Positives = 376/ |
| | 422 (88%), Gaps = 1/ | 422 (88%), Gaps = 1/ |
| | 422 (0%) | 422 (0%) |
| Mouse | — | Identities = 399/422 |
| (SEQ ID NO: 12) | | (94%), Positives = 412/ |
| | | 422 (97%), Gaps = none |

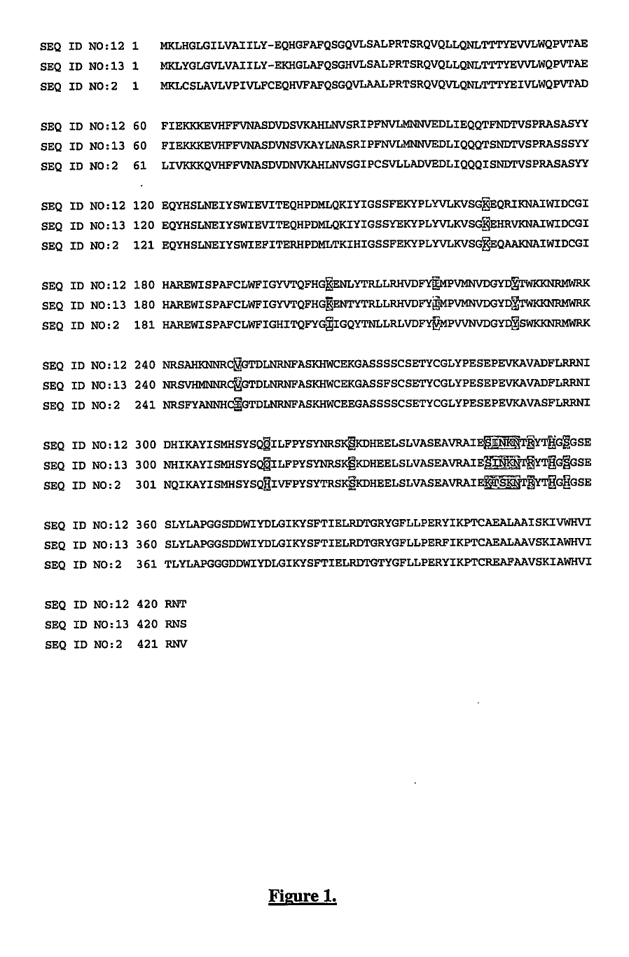

In view of the substantial sequence conservation between human rat and mouse CPU, the location of corresponding stabilizing mutations in the mouse (SEQ ID NO: 12) and rat (SEQ ID NO: 13) preproCPU are identified in FIG. 1 and FIG. 3.

1. A carboxypeptidase U (CPU) mutant polypeptide having greater thermal stability than the wild-type polypeptides, which mutant possesses at least two amino acid substitutions relative to the wild-type polypeptide, at least one of which is located at an amino acid residue position relative to SEQ ID NO: 2 selected from: 327, 355 and 357.

2. A carboxypeptidase U (CPU) mutant polypeptide according to claim 1, wherein at least two of the amino acid substitutions are selected from: 327, 355 and 357.

3. A CPU mutant polypeptide as claimed in claim 1, wherein there are at least 3 substitutions.

4. A CPU mutant polypeptide according to claim 1, which is a human polypeptide.

5. A CPU mutant polypeptide according to claim 1, which is a mouse or rat polypeptide.

6. A CPU mutant polypeptide according to claim 4, wherein at least one of the substitutions is selected from the group consisting of: S327C, H355Y, H357P and H357Q.

7. A CPU mutant polypeptide according to claim 6, wherein at least one of the substitutions is selected from the group consisting of: K166N, I204T, V219A, Y230C, I251T, H315R, S327C, K346N, S348N, K349R, N350S, R352K, H355Y, H357P and H357Q.

8. A CPU mutant polypeptide according to claim 1, which mutant possesses an amino acid substitution at each of positions: S327, H355 and H357, relative to SEQ ID NO:2.

9. A CPU mutant polypeptide according to claim 1 or claim 2, comprising the sequence selected from: SEQ ID NO: 17, 18 and 19.

10. A nucleic acid molecule encoding a polypeptide according to claim 1.

11. A nucleic acid molecule encoding a polypeptide according to claim 1 and a CPU prepro sequence.

12. A vector comprising a nucleic acid according to claims 10 or 11.

13. A cell comprising the nucleic acid according to claims 10 or 11.

14. A method of producing a CPU mutant polypeptide according to claim 1, comprising cultivating a cell according to claim 13, under conditions suitable to allow expression of the polypeptide and isolating the CPU mutant polypeptide produced.

15. A purified antibody, capable of selectively binding to a CPU mutant polypeptide according to claim 1.

16. A pharmaceutical composition comprising a therapeutically effective amount of the mutant CPU according to claim 1, and a pharmaceutically effective excipient or diluent.

17. A method of treating, preventing, managing or ameliorating the symptoms of hemorrhagic disease or disorder comprising administration of a therapeutically effective amount of a pharmaceutical composition according to claim 16.

18. A method of causing blood to clot comprising contacting the blood with an effective amount of a CPU mutant comprising the amino acid sequence according to SEQ ID NO: 2, but with at least two amino acid substitutions, at least one of which is at a position selected from the group consisting of: 327, 355 and 357.

19. A method of producing a crystal structure of a CPU mutant polypeptide according to claim 1, comprising allowing the polypeptide produced according to claim 14 to form a complex with a Fab fragment, purifying the complex and treating the purified complex under conditions suitable to allow crystal formation.

20. The method of producing wild-type CPU or proCPU crystals, comprising mixing together purified CPU or proCPU polypeptide with a Fab fragment directed to all or part of amino acids from positions 327 to 357 inclusive (according to the position in SEQ ID NO: 2) so as to allow complex formation, purifying the complex and treating the purified complex under conditions suitable to allow crystal formation.

21. A crystal of a mutant CPU polypeptide according to claim 1.


SEQUENCE LISTING (19 sequences)

SEQ ID NO: 1 - DNA, 1269 bp, Homo sapiens
atgaagctttgcagccttgcagtccttgtacccattgttctcttctgtgagcagcatgtcttcgcgtttcagagtggccaagttctagctgctcttcctagaacctctaggcaagttcaagttctacagaatcttactacaacatatgagattgttctctggcagccggtaacagctgaccttattgtgaagaaaaaacaagtccatttttttgtaaatgcatctgatgtcgacaatgtgaaagcccatttaaatgtgagcggaattccatgcagtgtcttgctggcagacgtggaagatcttattcaacagcagatttccaacgacacagtcagcccccgagcctccgcatcgtactatgaacagtatcactcactaaatgaaatctattcttggatagaatttataactgagaggcatcctgatatgcttacaaaaatccacattggatcctcatttgagaagtacccactctatgttttaaaggtttctggaaaagaacaagcagccaaaaatgccatatggattgactgtggaatccatgccagagaatggatctctcctgctttctgcttgtggttcataggccatataactcaattctatgggataatagggcaatataccaatctcctgaggcttgtggatttctatgttatgccggtggttaatgtggatggttatgactactcgtggaaaaagaatcgaatgtggagaaagaaccgttctttctatgcgaacaatcattgcatcggaacagacctgaataggaactttgcttccaaacactggtgtgaggaaggtgcatccagttcctcatgctcggaaacctactgtggactttatcctgagtcagaaccagaagtgaaggcagtggctagtttcttgagaagaaatatcaaccagattaaagcatacatcagcatgcattcatactcccagcatatagtgtttccatattcctatacacgaagtaaaagcaaagaccatgaggaactgtctctagtagccagtgaagcagttcgtgctattgagaaaactagtaaaaataccaggtatacacatggccatggctcagaaaccttatacctagctcctggaggtggggacgattggatctatgatttgggcatcaaatattcgtttacaattgaacttcgagatacgggcacatacggattcttgctgccggagcgttacatcaaacccacctgtagagaagcttttgccgctgtctctaaaatagcttggcatgtcattaggaatgtt

SEQ ID NO: 2 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKTSKNTRYTHGHGSETLYLAPGGGDDWIYDLGIKYSFTIELRDTGTYGFLLPERYIKPTCREAFAAVSKIAWHVIRNV

SEQ ID NO: 3 - DNA, 52 bp, Artificial
  Oligonucleotide Primer
tgctctagagcggccgcgggatgaagctttgcagccttgcagtccttgtacc

SEQ ID NO: 4 - DNA, 60 bp, Artificial
  Oligonucleotide Primer
atgatgatgcttatcgtcatcgtccccgggctcgagaacattcctaatgacatgccaagc

SEQ ID NO: 5 - DNA, 64 bp, Artificial
  Oligonucleotide Primer
cggggtaccttattaagatccactatgatgatgatgatgatgatgatgcttatcgtcatcgtcc

SEQ ID NO: 6 - DNA, 66 bp, Artificial
  Oligonucleotide Primer
ggggacaagtttgtacaaaaaagcaggcttcaccatgaagctttgcagccttgcagtccttgtacc

SEQ ID NO: 7 - DNA, 66 bp, Artificial
  Oligonucleotide
ggggaccactttgtacaagaaagctgggtcctaagatccactatgatgatgatgatgatgatgatg

SEQ ID NO: 8 - DNA, 18 bp, Artificial
  Oligonucleotide Primer
acccattgttctcttctg

SEQ ID NO: 9 - DNA, 20 bp, Artificial
  Oligonucleotide Primer
ttggtcttgctggaatcagt

SEQ ID NO: 10 - DNA, 57 bp, Artificial
  Oligonucleotide Primer
ccaagcttcatcccaacagcaattttctctagatctggtgaagctggagctacggag

SEQ ID NO: 11 - DNA, 18 bp, Artificial
  Oligonucleotide Primer
tgccaaaggggcggtccc

SEQ ID NO: 12 - protein, 422 aa, Mus musculus
MKLHGLGILVAIILYEQHGFAFQSGQVLSALPRTSRQVQLLQNLTTTYEVVLWQPVTAEFIEKKKEVHFFVNASDVDSVKAHLNVSRIPFNVLMNNVEDLIEQQTFNDTVSPRASASYYEQYHSLNEIYSWIEVITEQHPDMLQKIYIGSSFEKYPLYVLKVSGKEQRIKNAIWIDCGIHAREWISPAFCLWFIGYVTQFHGKENLYTRLLRHVDFYIMPVMNVDGYDYTWKKNRMWRKNRSAHKNNRCVGTDLNRNFASKHWCEKGASSSSCSETYCGLYPESEPEVKAVADFLRRNIDHIKAYISMHSYSQQILFPYSYNRSKSKDHEELSLVASEAVRAIESINKNTRYTHGSGSESLYLAPGGSDDWIYDLGIKYSFTIELRDTGRYGFLLPERYIKPTCAEALAAISKIVWHVIRNT

SEQ ID NO: 13 - protein, 422 aa, Rattus norvegicus
MKLYGLGVLVAIILYEKHGLAFQSGHVLSALPRTSRQVQLLQNLTTTYEVVLWQPVTAEFIEKKKEVHFFVNASDVNSVKAYLNASRIPFNVLMNNVEDLIQQQTSNDTVSPRASSSYYEQYHSLNEIYSWIEVITEQHPDMLQKIYIGSSYEKYPLYVLKVSGKEHRVKNAIWIDCGIHAREWISPAFCLWFIGYVTQFHGKENTYTRLLRHVDFYIMPVMNVDGYDYTWKKNRMWRKNRSVHMNNRCVGTDLNRNFASKHWCEKGASSFSCSETYCGLYPESEPEVKAVADFLRRNINHIKAYISMHSYSQQILFPYSYNRSKSKDHEELSLVASEAVRAIESINKNTRYTHGSGSESLYLAPGGSDDWIYDLGIKYSFTIELRDTGRYGFLLPERFIKPTCAEALAAVSKIAWHVIRNS

SEQ ID NO: 14 - DNA, 34 bp, Artificial
  Oligonucleotide Primer
atactcgagccaccatgaagctttgcagccttgc

SEQ ID NO: 15 - DNA, 39 bp, Artificial
  Oligonucleotide Primer
atcatgcggccgcttaaacattcctaatgacatgccaag

SEQ ID NO: 16 - protein, 20 aa, Artificial
  8-Histidine containing Peptide tag
LEPGDDDDKHHHHHHHHSGS

SEQ ID NO: 17 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKTSKNTRYTYGQGSETLYLAPGGGDDWIYDLGIKYSFTIELRDTGTYGFLLPERYIKPTCREAFAAVSKIAWHVIRNV

SEQ ID NO: 18 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASVVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKCKDHEELSLVASEAVRAIEKTNKNTRYTYGQGSETLYLAPGGGDDWIYDLGIKYSFTIELRDTGTYGFLLPERYIKPTCREAFAAVSKIAWHVIRNV

SEQ ID NO: 19 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKCKDHEELSLVASEAVRAIEKTSKNTRYTYGQGSETLYLAPGGGDDWIYDLGIKYSFTIELRDTGTYGFLLPERYIKPTCREAFAAVSKIAWHVIRNV